(19) United States
(12) Patent Application Publication
(10) Pub. No.: US 2005/0197546 A1
(43) Pub. Date: Sep. 8, 2005

(54) Security system

(76) Inventors: Eitan Mardiks, Ra'anana (IL); Yossi Bezalel, Herzelia (IL); Dan Ohad, Giv'ataim (IL)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/072,353

(22) Filed: Mar. 3, 2005

(30) Foreign Application Priority Data
Mar. 4, 2004 (IL) 160748

(51) Int. Cl. A61B 10/00; A61B 5/00; A61B 5/02; A61B 5/08

(52) U.S. Cl. 600/300; 128/920; 128/903

(57) ABSTRACT

A system for detecting irregular events in a monitored area by processing data related to the physiological parameters of guarding animals. The system comprises one or more sensors associated with one or more processing units for processing the physiological parameters by identifying and documenting, in real time, physiological patterns of the animals and comparing the generated physiological patterns with predetermined physiological patterns of the animal. The system further allows for extracting indications regarding the irregular events, whenever no match is obtained between the pattern or the generated patterns matching a pattern indicating an irregular event. The system also comprises communication means for transferring said physiological parameters from said animal to said one or more processing units.

### FIELD OF THE INVENTION

The present invention relates to the field of security systems. More particularly, the invention relates to a system for detecting irregular events in a monitored area by processing data related to the physiological parameters of animals, such as guard dogs.

### BACKGROUND OF THE INVENTION

Dogs are commonly used in the security market to intercept intruders or to interrupt a potential intruder operation. However, in the current situation, if the intruder neutralizes the guarding animal (e.g. by killing, drugging, or stealing it) there is no indication or notification that something happened to the animal. As a result this level of security is broken unawares.

Many privately raised pets are also used as guard dogs and serve as the first line of defense in the face of a hostile person trying to get into the protected territory. Nevertheless, an intruder may neutralize a watchdog relatively easily using poison, narcotics etc. The pet's owner has no indication of this type of action, rendering such a line of defense useless under these circumstances.

The level of security achieved by using a guard dog based system is, therefore, relatively low because of the simplicity of neutralizing the system. As a result, there is a need for the integration of a remote detection system to reduce the inherent risks of traditional security systems prior to their culminating in irreversible damage.

In the prior art such as GB 2,347,503, WO 02/37952 or U.S. Pat. No. 5,818,354, several devices were provided to remotely monitor the health of animals. However, none of these publications focus on the scenario within the security systems, in which a guard dog can be easily neutralized, e.g., by putting it into a sleep, thus these monitoring systems or devices will not be able to detect such a scenario. Therefore, they lack a crucial and necessary component to fulfill the security application they intend to provide.

All the systems described above have not addressed and have not yet provided satisfactory solutions to address the above described problem of significantly increasing the protection level achieved by using guard dogs.

It is an object of the present invention to provide a system for detecting irregular events in a monitored area with the aid of animals.

It is another object of the present invention to provide better means for monitoring the behavior and status of the animal in its guarding environment.

It is yet another object of the present invention to provide a system that is capable of handling a relatively large number of dogs and enable it to be integrated into existing security systems.

It is still another object of the present invention to provide a device which allows a pet owner to increase the level of flexibility and conduct a more anxiety-free lifestyle when having to leave a pet home alone especially after certain medical and/or surgical therapy or in cases of pet illness.

It is a further object of the present invention to provide a device which allows a veterinarian, animal hospital, animal shelter, pet store, or animal emergency service to provide better service to its customers by improving its animal monitoring capabilities.

Other objects and advantages of the invention will become apparent below.

### SUMMARY OF THE INVENTION

The present invention relates to a system for detecting irregular events in a monitored area by processing data related to the physiological parameters of an animal (such as respiratory signals, heart rate, pulse rate and intensity, motion, etc.) This comprises: a) one or more sensors for obtaining said physiological parameters; b) one or more processing units for processing said physiological parameters by generating, in real time, physiological patterns of said animal and comparing said generated physiological patterns with predetermined physiological patterns of said animal, and for extracting indicators regarding said irregular events, whenever no match is obtained between said pattern or said generated pattern matching a pattern indicating an irregular event; and c) communication means for transferring said physiological parameters from said animal to said one or more processing units.

According to a preferred embodiment of the invention, one or more sensors are installed or mounted on the animal, preferably—but non-limitatively, on a neck collar or a harness. In case an irregular event occurs (e.g., an intruder poisons the animal, shoots it, puts it to sleep or steals it) the signals representing the physiological parameters obtained by the one or more sensors are transmitted or transferred to the processing unit that analyzes the received transmitted signals and, if required, triggers an alarm. Preferably, at least one processing means is mounted on the animal, thus prior to being transferred to the final processing unit, signals are preprocessed by at least one said processing means.

According to a preferred embodiment of the present invention, the processing unit analyzes the pattern behavior and condition of the monitored animal in correlation to the measured parameters of one or more neighboring animals (e.g., behavior or condition, such as barking, physical activity or other physiological parameters). If the behavior of the neighboring animal(s) is above a predetermined threshold level an alarm signal will be generated, suggesting that an intruder is close to the monitored area. Preferably, the predetermined threshold level is based on previously collected data related to a specific animal.

Preferably, the alarm type is selected by the end user from a predefined menu consisting of, for example, an audible alarm, a silent remote message or any other type of notification method.

Preferably, the alarm or warning signal will also focus a camera or another type of sensor on the suspicious area for drawing the attention of on-duty personnel operating a control center, to the problem.

According to a preferred embodiment of the present invention, the system can simultaneously control several animals each identified by a unique ID.

### BRIEF DESCRIPTION OF THE DRAWINGS

The above and other characteristics and advantages of the invention will be better understood through the following illustrative and non-limitative detailed description of preferred embodiments thereof, with reference to the appended drawings, wherein:

FIG. 1 schematically illustrates a system for detecting irregular events in a monitored area by processing data related to the physiological parameters of an animal, according to a preferred embodiment of the present invention;

FIG. 2 schematically illustrates the mobile and stationary units of the system of FIG. 1;

FIG. 3 schematically illustrates the mobile unit of FIG. 1;

FIG. 4 schematically illustrates the stationary unit of FIG. 1;

FIG. 5 is a flowchart showing the process for detecting irregular events in a monitored area by processing data related to the physiological parameters of an animal, according to a preferred embodiment of the present invention;

FIG. 6 schematically illustrates an algorithm of the system of FIG. 1 for detecting irregular events in a monitored area by processing data related to the physiological parameters of single or multiple animals, according to a preferred embodiment of the present invention;

FIGS. 7A and 7B schematically illustrate the Respiratory Cycle of an animal.

### DETAILED DESCRIPTION OF PREFERRED EMBODIMENTS

The invention will now be described primarily with reference to the examples of a security system. These examples are provided merely to illustrate the invention and are not intended to limit its scope in any way. It should be understood that the invention can be applied mutatis mutandis to other applications such as veterinary facilities, private pet owners, pet stores, animal shelters, etc.

The term “irregular events” refers to any activity or situation in the monitored area or its related surroundings, such as the approach of hostile person(s) to the monitored area, a sudden noise, etc. that may cause the animal to change its behavior, condition or position.

The system of the present invention utilizes the activity of guard dogs to detect irregular events in a monitored area by processing data related to the physiological parameters of each dog and report in case the dog is not functioning as it should be. The system further provides a warning signal in case the dog's behavior (and optionally in correlation to data gathered from neighboring dogs' parameters) demonstrates that an intruder is coming close. The system of the present invention is capable of handling a group of dogs and is capable of being integrated to existing security systems.

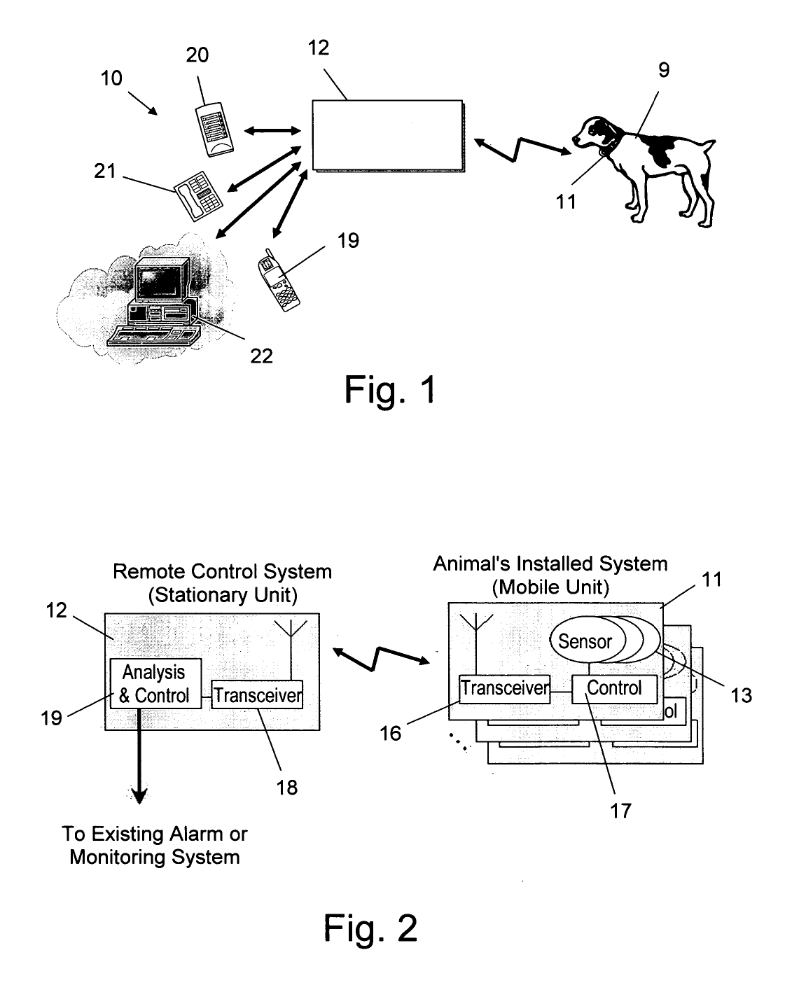

FIG. 1 schematically illustrates a system for detecting irregular events in a monitored area by processing data related to the physiological parameters of an animal, according to a preferred embodiment of the present invention. System 10 comprises a mobile Unit 11 that is attached to Animal 9 (e.g., to its collar or harness) and a stationary unit, # 12. Mobile unit 11 comprises one or more sensors 13 (FIG. 2) to obtain physical parameters regarding the animal's health status, and a transceiver 16 (FIG. 2) to transmit the obtained parameters to the stationary unit 12. Mobile unit 11, may further comprise a control unit 17 (FIG. 2) to perform a basic analysis of the sensor output (i.e., of the obtained parameters).

Preferably, a given sensor such as 13 can be selected from a variety of sensors to be optimized for various kinds of animals or for various applications. For example, Sensor 13 can be a microphone or piezoelectric device monitoring the respiration and heart beat of the animal. In order to provide information about the condition of the animal's health. Another type of sensor is a motion detector (based on the accelerometer). This type of sensor provides information about the animal's activity level associated with other signs.

The stationary unit 12 receives the data representing the physical parameters from mobile unit 11 and performs an analysis of the data to gather the most reliable input regarding the animal's status in real time, and as a result helps decide whether to report the appearance of an irregular event, and optionally, how to report it (such as to a cellular phone 19, to an existing security system 20, to a telephone 21, PC 22 via the internet, etc).

The stationary unit 12 (FIG. 2) is capable of collecting data from more than one mobile unit 11 (i.e., from several animals) as well as from an external system (e.g. an existing security system, a data base, etc.), process and analyze it, and store descriptive statistical information. The stationary unit 12 is also capable of self adjusting, retuning its own parameters based on comparison to data collected previously from same animals and adapting itself to changes over time (i.e., possessing a learning mechanism).

This mobile unit 11 is operated by a power source, such as a battery (not shown). The stationary unit 12 is connected to a power source (either internal or external) and may optionally contain a rectangular backup battery (not shown). Unit 12 can be a stand alone unit, be added on to an existing alarm system, or be an accessory for an existing PC (e.g. connected via a USB port) or a phone (wireless or wired).

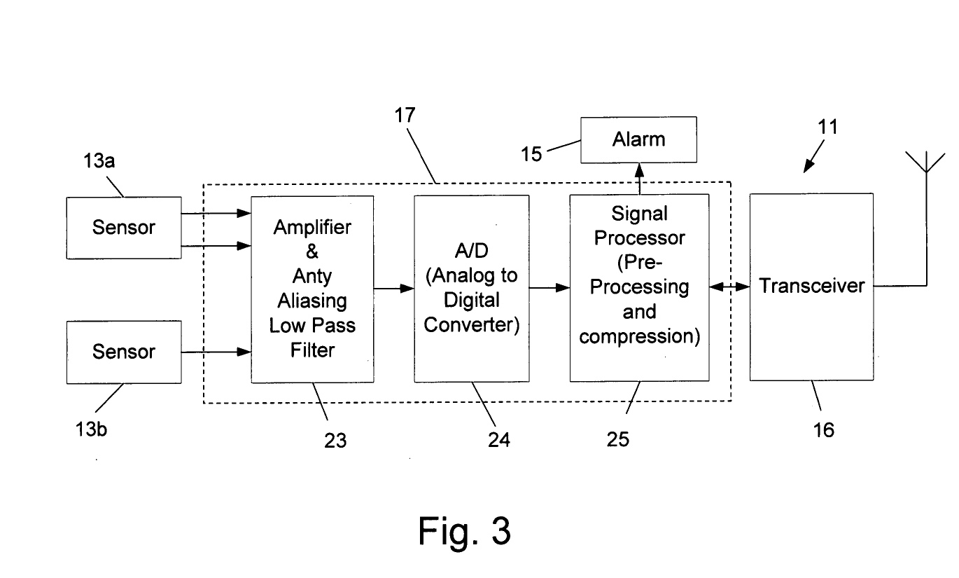

FIG. 3 schematically illustrates the mobile unit 11, according to one preferred embodiment of the invention. Mobile unit 11 comprises several types of sensors 13a and 13b, control unit 17, transceiver 16 and optionally, alarm unit 15 for warning or notifying of irregular events. The sensors may be one or more microphones 13a and/or other types of sensors used for monitoring respiratory activity (i.e., first type of sensors) and a motion sensor 13b (i.e., second type of sensor) which can be used for reporting the animal's motion frequency, pattern, rate and rhythm. The sensors' input signals are sampled and transferred to control unit 17 that amplifies and performs pre-processing and compression of the signals to reduce the amount of the transmitted information. To keep the mobile parts as small as possible, as well as to extend the battery life span to a maximum, tasks performed by the mobile unit will be kept at a minimal level.

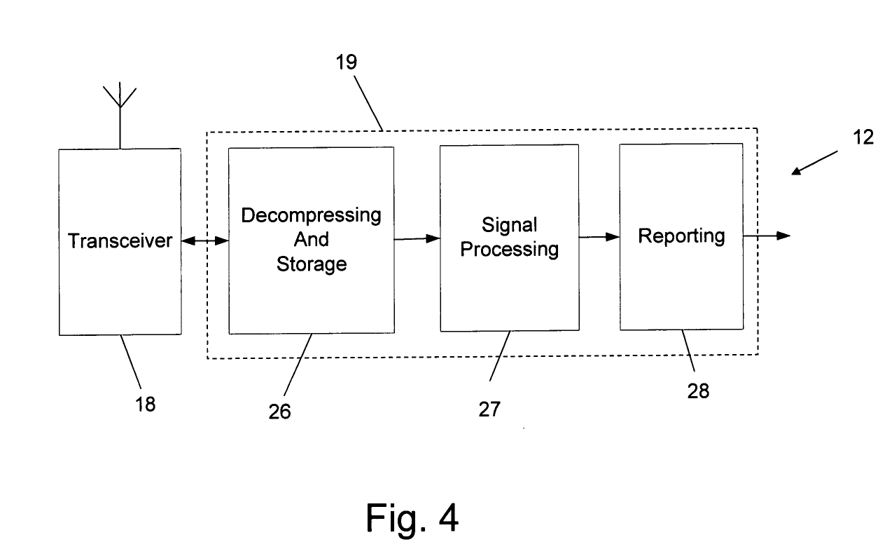

FIG. 4 schematically illustrates the stationary unit 12, according to a preferred embodiment of the invention. Stationary unit comprises transceiver 18 and analysis and control unit 19. The signal-processing tasks are performed by unit 19. The stationary unit 12 decompresses (unit 26) the signals received from mobile unit 11, performs their analysis (unit 27) and reports (unit 28) via a desired or predefined method, such as via a cellular network to a cell phone, beeper, pager, alarm system, PC, internet or other communication methods, etc.

Stationary unit 12 is used for processing the data representing the physiological parameters of the animal by generating, in real time, physiological patterns or trends of the animal and continuously comparing the recorded physiological pattern to previously acquired patterns. The memorized physiological patterns used for comparison are stored in a storage means related to control unit 19, such as in unit 26. They predetermined physiological patterns can reflect any physiological pattern of the animal, such as the respiration rate of the animal while it is asleep, runs, eats, etc. or it can be also a physiological pattern that was measured or recorded while the animal was frightened by a sudden noise (e.g., a shot-gun). The latter may represent a predetermined pattern which may occur during an irregular event. Unit 12 extracts indicators regarding irregular events, whenever no match is obtained between the generated pattern in real time and the predetermined ones, or when the generated pattern does match a previously memorized pattern identified as indicative of an irregular or threatening event.

Optionally, the algorithm (e.g., firmware and/or software) installed within stationary unit 12 and/or within the mobile unit 11 may be updated via any applicable communication means, such as the Internet, a dedicated phone line etc, including remotely.

The mobile unit 11 is used to gather several parameters in order to aid system 10 to determine whether an irregular event occurs. The gathered physiological parameters can be analyzed both in the mobile unit 11 and in the stationary unit 12. Preferably, but non-limitatively, the measured physiological parameters are:
Vocalization
Respiration pattern, as described hereinafter;
Respiration rate;
Respiration sounds;
Heart rhythm;
Heart rate;
Heart sounds;
Electrocardiographic data;
Body temperature;
Body acceleration
Limb motion or movement;
Electric Skin Conductivity;
Animal's hair erection; and
Muscle tone (tension).

In addition to these parameters, system 10 is also capable of sending stimulating signals to the animal to test its awareness to its surroundings. Such a stimulus may be a sound, a weak electrical shock, etc.

According to a preferred embodiment of the present invention, using the aforementioned parameters provides enhanced means for monitoring the behavior and status of the animal in its guarding environment, in particular while analyzing the respiration pattern of the guarding animal. Analyzing those parameters allows the determination of whether a guard dog has been neutralized, such as if it has been put to sleep, stolen or neutralized in other ways. As a result of using such parameters, the system of the present invention provides a sensitive way to remotely detect sudden circumstantial changes experienced by a guard animal, through remote documentation of the physiological impact elicited by such external or internal stimuli. Using a specific algorithm which analyzes those parameters may enable the detection of and warning about a hostile person approaching the animal, by reflecting consequent behavioral or physiological changes of the animal.

Scientific Background—Circumstantial Changes as Experienced by a Guarding Animal

The adrenergic effect related to stimuli such as excitement, fear, pain or similar sudden stressors in animals is well known to be mediated initially through the sympathetic nervous system. If stress persists, hypothalamic secretion of corticotrophin releasing factor stimulates the release of hormones such as Adrenocorticotrophic Hormone (ACTH) from the anterior pituitary gland. ACTH, in turn, causes an increase in secretion of corticosteroid hormones from the adrenal gland into the circulatory system. These Neurohumoral fluctuations assist short and long-term physiological adaptation of the animal to the applied stress.

The gathering of the aforementioned parameters is based on the fact that mammals in general and canines in particular, exhibit measurable physiological responses to a variety of acute (sudden) environmental stimuli that may be subjectively experienced by them as stressors. This variation in potentially stressful external conditions spans over a large spectrum, including, on the one end, extreme, genuinely life-threatening conditions, and on the other end, mild conditions that are hardly even experienced (if at all) by the conscious, aware animal.

Stimuli such as pain, heat, emotional or behavioral anxiety, loud and sudden sounds, a flash light in the dark, yells or screams of intruders into the canine's perceived territory, etc., trigger an increase in the sympathetic nervous activity and in plasma levels of hormones such as ACTH and cortisol. Animal responses to such stressful circumstances may be detected by changes in respiratory rate, heart rate, blood pressure, plasma cortisol and plasma ACTH. For example, in chronically instrumented dogs awake and breathing spontaneously at rest and during a mild dry heat stress, the resting respiratory rate increased from 16.9 to 192.8 breaths/min (or 3.2 Hz).

Some such adaptive responses, however, e.g. respiratory ones, do not require consciousness of the animal. The above-described conditions are likely located somewhere in the center of the spectrum of stressful circumstances. Nonetheless, similarly measurable physiological responses have been documented in the two extreme points at the two opposing ends of that same spectrum, whether animals actually experienced no danger as they were unconscious due to artificially induced sleep or whether they were already unconscious as they were in the process of dying due to extreme, lethal environmental conditions.

Examples for potentially stressful circumstances that may reflect very different points anywhere along the above-mentioned spectrum include a sudden physical insult (e.g. a gun shot, an orally introduced poisonous bait, a lethal or an anesthetic spray, a sedative or a tranquillizer injected intramuscularly via a dart which takes its pharmacological effect only after having elicited a stressful experience in the animal), to a guarding or to a pet dog.

Objective measurement of trends of change in canine physiological parameters can reflect their being subject to such abrupt changes in environmental circumstances that may or may not be subjectively experienced by them as potential stressors.

According to a preferred embodiment of the present invention, in order to evaluate the physiological effects of canines during sudden circumstantial changes that could represent any point along the above mentioned spectrum of stressful conditions, an algorithm for evaluation the results of neural and endocrine alterations indirectly is used, preferably, by focusing on the analysis of changes in the respiratory rate parameter. This parameter is relatively more sensitive and amenable for obtaining a reliable, non-invasive monitoring while using sensors such as microphones directed towards the ventral section of the cervical trachea.

According to a preferred embodiment of the present invention, system 10 provided with one or more algorithms for continuously monitored and averaged specific parameters throughout the Respiratory Cycle. These specific parameters throughout the Respiratory Cycle can include:
1. Inspiratory phase duration (which can change with and influence the Tidal Volume); and 2. Expiratory phase duration
   Definitions:

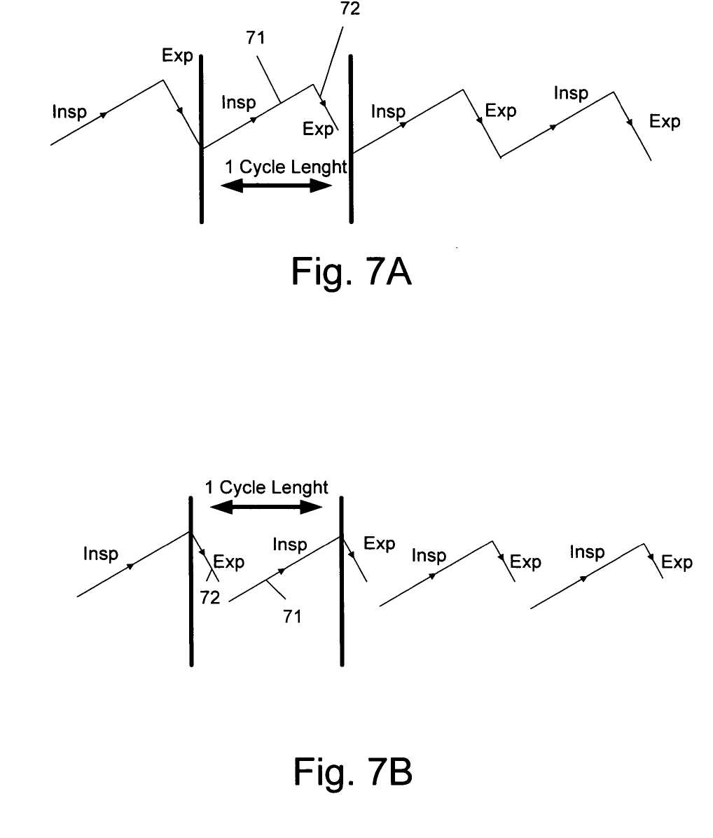

The sum of both inspiratory phase duration and expiratory phase duration reflects a respiratory cycle. FIG. 7 schematically illustrates a representation of respiratory cycles, wherein line 71 indicates the inspiratory phase duration and line 72 indicates the expiratory phase duration. Each pair of lines such as 71 and 72 indicates one respiratory cycle length.

The Respiratory Rate is the number of cycles (either end-inspiration-to-end-inspiration, or end-expiration-to-end-expiration) per minute (which is one component determining the minute ventilation (the volume of air breathed over a period of one minute, and a potentially direct measure of excitement levels.

The Inspiratory-to-Expiratory time ratio (the ratio between Inspiratory phase duration and Expiratory phase duration within each respiratory cycle influences oxygen partial pressure in arterial blood (PaO₂), carbon dioxide partial pressure in arterial blood (PaCO₂), and serum pH levels (e.g. the higher the ratio, the higher the PaCO₂ and the lower the pH, and vice versa).

Should animals be continuously subjected to high ambient temperatures and humidity, the resting respiratory rate is expected to be relatively very high (as in panting). According to a preferred embodiment of the present invention, system 10 will regard this resting respiratory rate as normal background since it will be constantly learning normal behavior via monitoring and documentation of a running average of all of the above parameters, updating that average on a regular basis and in real time. Any physiologically and/or statistically significant change (whether an increase or a decrease) from that running average (as predefined by a user) will trigger an alarm signal according to predetermined parameters that can be flexibly changed according to specific recommendations provided by the manufacturer of such system, type of animal, to needs, or to user judgment.

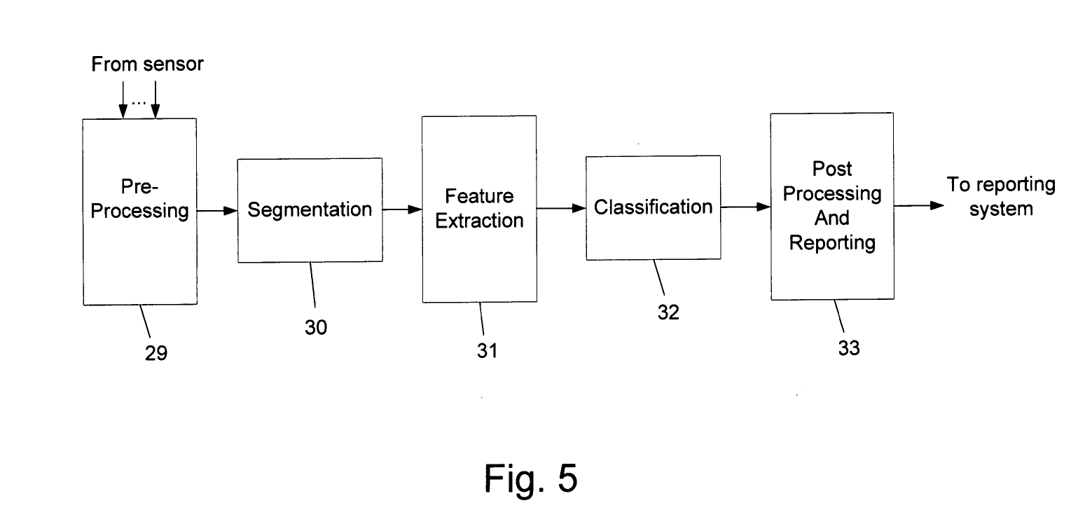

The algorithms of the present invention are implemented in mobile unit 11 and stationary unit 12. FIG. 5 is a block diagram showing the process of monitoring animals, according to one algorithm of the present invention. The following described algorithm applies to the whole system 10 (mobile 11 and stationary 12 units). The first operation of the algorithm is referred to the analysis which occurs at the mobile unit 11 (i.e., pre-processing), and is intended to “clean” received signals from the sensors and remove noise (block 29), e.g., performing digital filtering of sampled signals, as known to trained operating personnel. The second operation of the algorithm takes place at the stationary unit 12, analyzing the received signal and divides it into several well-defined objects (block 30). At the next step, feature extraction (block 31) the algorithm analyzes the objects and identifies the relevant features of each object.

At the next step, block 32, the classification block, is relatively the most critical and sophisticated part of the algorithm. This block classifies the object into groups; each group is related to an output to be reported (e.g. color-coded animal condition such as “Green”, “Yellow” or “Red”). This classification can be based on any suitable statistical methods and/or a neural network algorithm, as is known to a person skilled in the art.

The output of the classification block 32 is fed into a post processing and reporting unit, block 33, that constructs the appropriate reporting message (sound, speech, text, alarm trigger etc.) and encapsulates it using the format of choice according to the reporting unit in use.

The system can be fit to a wide range of applications and animal types, wherein most of the adaptations to the specific animal type or applications are controlled by dedicated algorithms or software. For example, the system can differentiate between normal (natural and spontaneously occurring sleep and artificially induced (chemically forced) sleep resulting from drugging the animal. This difference can be recognized by utilizing the fact that there are two clinically noticeable differences between natural sleep and artificial sleep (i.e. general anesthesia) in healthy animals. According to a preferred embodiment of the present invention, system 10 monitors and analyzes both differences by one or more algorithms based on the following facts:
1) During natural sleep, animals (as do human beings) tend to remain motorically active and move around from time to time, changing their body position and “maintain their level of comfort”, despite being totally unaware of this activity due to being unconscious at that specific time. This is not reported in artificially sleeping animals when under general anesthesia.
2) During natural sleep, there is periodically a deeper and longer inhalation phase (similar to a yawn or a sigh), which is absent during general anesthesia. So much so, that anesthetists are trained to “sigh” for their anesthetized patients from time to time, by periodically “bagging” their lungs (i.e. inflating their lungs with a larger amount of anesthetic gas-volume along with more oxygen), and document the exact timing of this activity in the anesthetic log, to prove it has been done in a timely fashion. A naturally sleeping animal is able to do this spontaneously even when asleep, and needs no such external, artificial support at any point in time during its natural sleep. In contrast, an animal that was chemically put to sleep shows no such activity throughout the time it remains unconscious until recovery from anesthesia.

Taking advantage of these two simple differences between natural and artificially induced sleep, the system will be capable of identifying un-natural loss of consciousness such as induced by intramuscularly injected anesthetic agents.

According to a preferred embodiment of the present invention, the system is further provided with an adaptive learning mechanism applied to increase system reliability over time, by utilizing a suitable statistical methods and/or a neural network algorithm, well known to trained personnel.

According to a preferred embodiment of the present invention, System 10 further comprises an algorithm for comparing the behavior and/or health condition of each animal with its neighboring animals, to enhance the decision making process regarding the condition and status of a specific animal, thereby aiding to determine whether an unusual behavior of one or more animals occurs due to intrusion. Such enhancement is achieved by identification of abnormal physiology in a group of neighboring animals, confirming that such a change in any single animal of this specific group is less likely to be coincidental. Further confirmation is achieved by a “moving front” of such changes if identified among different neighboring animals, as an intruder-to-be patrols around them in a certain direction, an action that will be readily identified by the system as it follows the “wave of physiological change” in animals facing that mobile intruder.

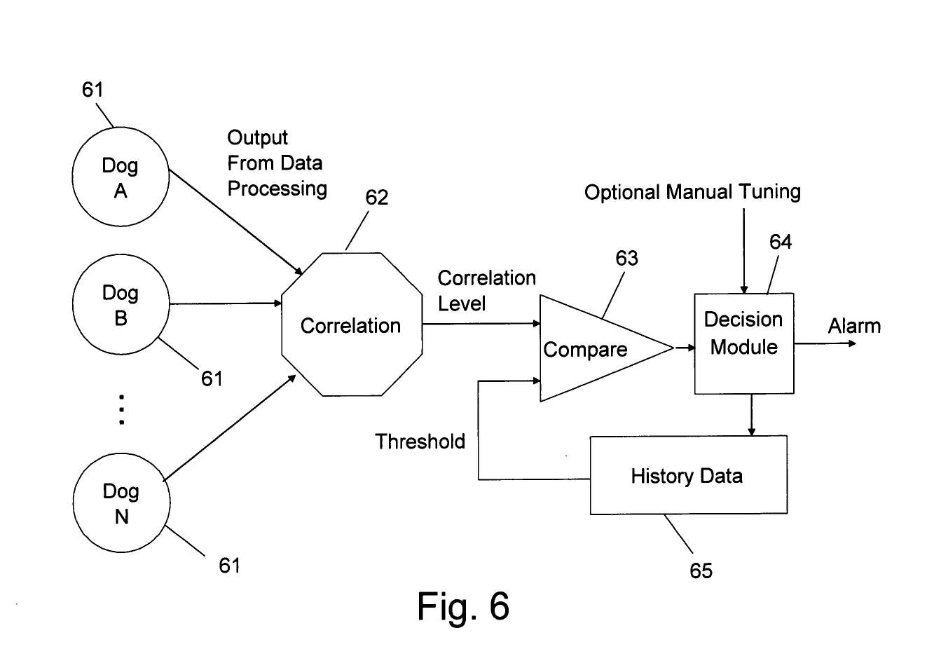

FIG. 6 schematically illustrates an algorithm for detecting unordinary events in a monitored area by processing data related to the physiological parameters of single or multiple animals, according to a preferred embodiment of the present invention. In order to detect unordinary events, the algorithm performs the following steps:
First (Blocks 61), pre-processed data are obtained from the sensors mounted on each animal;
Next (Block 62), the processing unit of stationary unit 12 analyzes the pattern behavior and condition of each monitored animal in correlation to the measured parameters of one or more neighboring animals (e.g., behavior or condition, such as barking, physical activity or other physiological parameters), and in response, generating data representing a specific correlation level;
Next, (Block 63), the system compares the generated correlation level with a predetermined threshold level. Preferably, the predetermined threshold level is based on previous collected data (Block 65) related to each animal;
Next (Block 64), a decision module of the algorithm receives the compared results and if the behavior of the neighboring animal(s) is interpreted as reflecting a level of alertness beyond a predetermined threshold an alarm signal is generated, (e.g., suggesting that an intruder is close to the monitored area). Optionally, Block 64's decision module can be manually fine-tuned or adjusted. For example, to increase the systems sensitivity and specificity (i.e. to avoid both missed and false alarms) the threshold for an alarm triggered by changes in the physiological status of an individual animal can be automatically lowered pending detection of change in neighboring animals, and vice versa.

As illustrated in FIG. 1 the stationary unit 12 can communicate with a wide range of system types, such as a home security system, a personal computer, a mobile phone, etc.

The system of the present invention can also be used by a veterinarian for remotely watching hospitalized animals or by other organizations that keep, maintain, guard, oversee, or host animals for any other reasons or purposes.

The system of the present invention reports in real time any changes in the physiological status of animals. The system can be used in a variety of applications, such as airports, military bases, power stations, prisons, residential areas and other facilities using dogs as a part of their security concept, as well as other institutions that keep guard dogs for extra protection. The system will provide an added value to any of these customers by providing better protection. The system can send an alarm either only to a central station or activate an existing local security system in any and/or all individual components of a given facility.

In addition, the system of the present invention can also be used for other application, such as private pets that are also being used for security purposes outdoors or indoors, or if it just being held outside of the house. For example, if some hostile person is neutralize stolen or harm the guard dog the system can activate an alarm or send a message to the owner or to a security center notifying that something wrong happening.

An owner of a sick pet needing intensive attention or care, or an owner merely interested in better and closer monitoring of his/her pet even when unseen will be able to get a real time warning message to a mobile phone, land phone, beeper, or similar device, whenever the pet's well-being is compromised, whether indoors or outdoors. Using the system of the present invention may provide pet owners reassurance and a better sense of security when leaving their pets unattended.

A vet that keeps animals in his clinic might use the system to notify him in case of emergency with one of the animals.

Another application for the system is a veterinary one. Upon alarm triggered by the system and identified at a central location, on-call personnel will be prompted to arrive to the pet location (a veterinary hospital or an owner's residence) and provide emergent care at a timely fashion.

Animal kennels may use the system for better and closer monitoring and care for kenneled animals, thereby upgrading its reputation among customers.

The system of the present invention allows using a device that is simply attaches to the pet by a collar or a harness, and transmits a warning message to the owner or to a call center via the stationary unit 12. Using such a device allows a pet owner to:
Be more flexible and less anxious about leaving a pet unattended, especially following certain medical treatments or in the case of chronic illness.
Be more confident about leaving his pet at a veterinary facility that uses a dedicated monitoring device for hospitalized pets left for close watch in the clinic.
A vet (or other special service company) will be able to provide a call center service and rush to a pet left home alone in case of a serious heath problem of the pet.
Small veterinary clinics that have no 24 hour watch might use such a device to alert them to rush to the clinic in cases where a pet is in serious condition. Kennels for example might provide better service having a closer watch of its residents.

The above examples and description have been provided only for the purpose of illustration, and are not intended to limit the invention in any way. As will be appreciated by the skilled person, the invention can be carried out in a great variety of ways, employing more than one technique from those described above, all without exceeding the scope of the invention.

1. A system for detecting irregular events in a monitored area by processing data related to the physiological parameters of animal(s), comprising:
a) one or more sensors for obtaining said physiological parameters;
b) one or more processing units for processing said physiological parameters by identifying and documenting, in real time, physiological patterns of said animal and comparing said generated physiological patterns with predetermined physiological patterns of said animal, and for extracting indications regarding said irregular events, whenever no match is obtained between said pattern or said generated pattern matching a pattern indicating an irregular event;
c) Communication means for transferring said physiological parameters from said animal(s) to said one or more processing units.

2. A system according to claim 1, further comprising means for reporting of a condition change.

3. A system according to claim 2, in which the means for the reporting of a condition of change are communication means for sending an alarm or warning signals.

4. A system according to claim 1, in which the one or more sensors are installed or mounted on a collar or a harness of an animal.

5. A system according to claim 1, in which at least one processing means, is mounted on the animal, such that prior to being transferred, signals are preprocessed by at least one said processing means.

6. A system according to claim 1, in which the processing unit analyzes the behavior and condition patterns of monitored animals as a stand-alone event or in correlation to the behavior or condition of at least one other animal, such that whenever the level of alertness of any of the animals is above or below predetermined threshold levels as compared to previously collected data from same animals, an alarm signal is generated.

7. A system according to claim 6, in which alarm signal types are selected from a menu consisting of audible alarms, remote messages, or any other type of notification method or combination thereof, as elected by end users.

8. A system according to claim 6, in which the alarm signal focuses a camera or another type of sensor on the suspicious area.

9. A system according to claim 1, in which said system simultaneously controls large numbers of animals each identified by a unique ID.

10. A system according to claim 1, in which the physiological parameters are selected from a group consisting of vocalization, respiration pattern, respiration rate, respiration sounds, heart rhythm, heart rate, heart sounds, electrocardiogram, body temperature, body acceleration, limb motion or movement, electrical skin conductivity, hair erection, or muscle tone (tension).

11. A system according to claim 1, further comprising an adaptive learning mechanism for increasing the system reliability over time, by utilizing suitable statistical methods and/or a neural network algorithm, and/or other methods.

12. A system according to claim 1, further comprising means for sending stimulating signals to the animal to test its awareness to the environment.

13. A system according to claim 1, in which the communication means are also, used for remote software and/or firmware updates of said system.

14. A system according to claim 1, further comprising means for detecting the approach of intruders or hostile person(s) to the animal.

15. A system according to claim 1, further comprising means for sending data to a manned call-center.

16. A system according to claims 3, in which the alarm or warning signal is generated in correlation with the animal's behavior, and optionally in correlation with the neighboring dogs' parameters, whenever said system decides that an intruder is approaching.

17. A system according to claim 1, further comprising means for handling more than one animal simultaneously.

18. A system according to claim 1, in which the communication means are capable of communication with other existing security systems, thereby integrating with said existing security systems.

19. A system according to claim 1, in which the parameters allow determining whether one or more animals are neutralized or over-excited, thereby said system allows remote detection of sudden circumstantial changes as experienced by said animals, through remote documentation of the physiological impact elicited by external or internal stimuli.

20. A system for detecting irregular events in a monitored area by processing data related to the physiological parameters of animals.

21. A system according to claim 1, in which said physiological parameters include differentiations between natural sleep and chemically induced sleep by monitoring body position changes or irregularity of respiratory cycles.

22. A system according to claim 6, in which the said parameters include comparing the pattern of a group of animals for identifying a sequential spreading change among the members of the group

23. A system according to claim 6, in which the pattern of behavior of any given animal of the group is correlated relative to the pattern of other animals of the same group as a function of said given animal characteristic behavior.
